(19) United States
(12) Patent Application Publication
(10) Pub. No.: US 2024/0382697 A1
(43) Pub. Date: Nov. 21, 2024

(54) BLOOD VESSEL VISUALIZATION DEVICE, BLOOD VESSEL PUNCTURE SYSTEM, AND BLOOD VESSEL VISUALIZATION SYSTEM

(71) Applicant: TERUMO KABUSHIKI KAISHA, Tokyo (JP)

(72) Inventor: Shota TSUMAKI, Kai-shi (JP)

(73) Assignee: TERUMO KABUSHIKI KAISHA, Tokyo (JP)

(21) Appl. No.: 18/787,637

(22) Filed: Jul. 29, 2024

Related U.S. Application Data
(63) Continuation of application No. PCT/JP2023/003490, filed on Feb. 3, 2023.

(30) Foreign Application Priority Data
Feb. 9, 2022 (JP) 2022-018463

(51) Int. Cl. A61M 5/42

(52) U.S. Cl. CPC A61M 5/427; A61M 2205/02; A61M 2205/3306; A61M 2205/502; A61M 2205/8206; A61M 2209/088; A61M 2210/08; A61M 2210/12

(57) ABSTRACT

A blood vessel visualization device for visualizing a blood vessel of a living body in a site to be visualized that includes a first surface and a second surface on a side opposite the first surface, includes: an attachment portion attachable to and detachable from the site to be visualized; and an irradiation unit including a light source unit for irradiating the first surface with near-infrared light. The attachment portion includes a wavelength converter including a wavelength conversion material that converts the near-infrared light into visible light, the attachment portion being fixable to the site to be visualized in a state in which the wavelength converter is in contact with the second surface.

### CROSS-REFERENCE TO RELATED APPLICATIONS

This is a bypass continuation of PCT Application No. PCT/JP2023/003490, filed on Feb. 3, 2023, which claims priority to Japanese Patent Application No. JP2022-018463, filed on Feb. 9, 2022. The entire contents of these applications are incorporated herein by reference.

### BACKGROUND

### Technical Field

The present disclosure relates to a blood vessel visualization device, a blood vessel puncture system, and a blood vessel visualization system.

### Related Art

For example, JP 2017-64094 A discloses a vein visualization device. The vein visualization device includes an irradiation unit, an imaging unit, an image processing means, and a display unit. The irradiation unit irradiates a site to be punctured of a patient with near-infrared light. The imaging unit receives reflected light resulting from reflection of the near-infrared light emitted to the site to be punctured on the site to be punctured and acquires a captured image of the site to be punctured. The image processing means extracts a vein from the captured image. The display unit displays the image processed by the image processing means.

### SUMMARY

The above-described vein visualization device requires a member that converts near-infrared light into an image, and thus, entails a problem of having a large and complicated configuration.

An object of the present disclosure is to solve the above problems.

(1) A first aspect of the present disclosure provides a blood vessel visualization device for visualizing a blood vessel of a living body, the blood vessel visualization device including: an attachment portion attachable to and detachable from a site to be visualized; and an irradiation unit including a light source unit that irradiates a first surface of the site to be visualized with near-infrared light, wherein the attachment portion includes a wavelength converter including a wavelength conversion material that converts the near-infrared light into visible light, the attachment portion being fixable to the site to be visualized in a state in which the wavelength converter is in contact with a second surface of the site to be visualized on a side opposite to the first surface.

(2) In the blood vessel visualization device according to (1), it is preferable that the attachment portion includes an attachment base that is in contact with the first surface in a state in which the attachment portion is attached to the site to be visualized.

(3) In the blood vessel visualization device according to (2), it is preferable that the wavelength converter is removably mounted on the attachment base.

(4) In the blood vessel visualization device according to (2) or (3), it is preferable that the light source unit is mounted on the attachment base.

(5) In the blood vessel visualization device according to (4), it is preferable that the light source unit is mounted on an inner surface of the attachment base.

(6) In the blood vessel visualization device according to (4) or (5), it is preferable that the irradiation unit includes a power supply unit that supplies power to the light source unit, and the power supply unit is mounted on the attachment portion.

(7) In the blood vessel visualization device according to any one of (1) to (3), it is preferable that the irradiation unit is provided separately from the attachment portion.

(8) In the blood vessel visualization device according to any one of (1) to (7), it is preferable that the wavelength converter is molded into a predetermined shape with a material containing the wavelength conversion material.

(9) In the blood vessel visualization device according to any one of (1) to (7), it is preferable that the wavelength converter includes: a base including a material not containing the wavelength conversion material; and a coating formed by coating a surface of the base with the wavelength conversion material.

(10) In the blood vessel visualization device according to any one of (1) to (9), it is preferable that the wavelength conversion material converts the near-infrared light having a wavelength of more than 700 nm and 2500 nm or less into the visible light having a wavelength of 400 nm or more and 700 nm or less.

(11) In the blood vessel visualization device according to any one of (1) to (10), it is preferable that the wavelength converter has a puncture hole for puncturing the blood vessel with a medical device.

(12) In the blood vessel visualization device according to any one of (1) to (11), it is preferable that the attachment portion has a glove shape.

(13) A second aspect of the present disclosure provides a blood vessel puncture system including: the blood vessel visualization device according to any one of (1) to (12); and a medical device that punctures the blood vessel.

(14) A third aspect of the present disclosure provides a blood vessel visualization system including: the blood vessel visualization device according to any one of (1) to (12); a camera that captures a blood vessel image displayed on the wavelength converter; and an image processing unit that analyzes an image captured by the camera.

According to the present disclosure, near-infrared light is emitted from the light source unit to the site to be visualized in a state in which the attachment portion is attached to the site to be visualized. Thus, the near-infrared light passes through a region of the site to be visualized other than the blood vessel. In other words, the near-infrared light is absorbed by hemoglobin in the blood in the blood vessel at the site to be visualized. Transmitted light of the near-infrared light transmitted through the site to be visualized is converted into visible light by the wavelength conversion material of the wavelength converter. Therefore, a visible blood vessel image is displayed on the wavelength converter. That is, the blood vessel visualization device can display the blood vessel image on the wavelength converter located on the site to be visualized without using a member that converts the near-infrared light into an image. Therefore, the blood vessel can be visualized with a compact and simple configuration.

In addition, the wavelength converter can be fixed to the site to be visualized in a state of being in contact with the second surface of the site to be visualized, whereby the near-infrared light transmitted through the site to be visualized can be efficiently received by the wavelength converter. Therefore, the blood vessel image can be more clearly displayed on the wavelength converter.

### BRIEF DESCRIPTION OF DRAWINGS

FIG. 1 is an explanatory diagram schematically illustrating a configuration of a blood vessel visualization system according to an embodiment of the present disclosure;

FIG. 2A is a bottom view of the blood vessel visualization device in FIG. 1;

FIG. 2B is a plan view of the blood vessel visualization device in FIG. 1;

FIG. 3 is an explanatory cross-sectional view taken along line III-III in FIG. 2B;

FIG. 4 is an explanatory diagram schematically illustrating a configuration of a blood vessel visualization device according to a modification;

FIG. 5A is a schematic bottom view of the blood vessel visualization device in FIG. 4;

FIG. 5B is a plan view of the blood vessel visualization device in FIG. 4;

FIG. 6 is an explanatory cross-sectional view taken along line VI-VI in FIG. 5B;

FIG. 7 is an explanatory diagram schematically illustrating a configuration of a blood vessel puncture system according to an embodiment of the present disclosure; and

FIG. 8 is an explanatory cross-sectional view of a wavelength converter according to a modification, a part of which is omitted.

### DETAILED DESCRIPTION

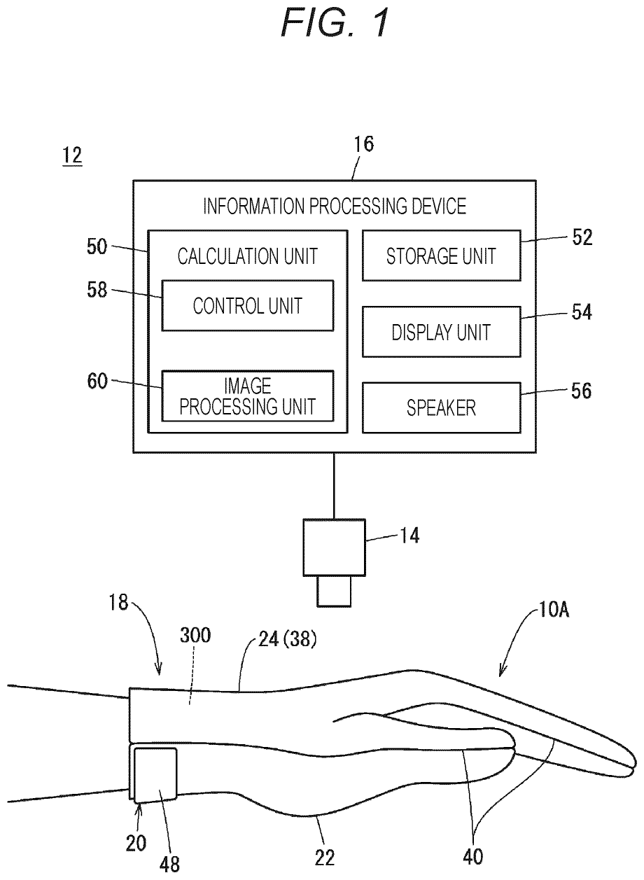

As illustrated in FIG. 1, a blood vessel visualization system 12 according to an embodiment of the present disclosure includes a blood vessel visualization device 10A, a camera 14, and an information processing device 16.

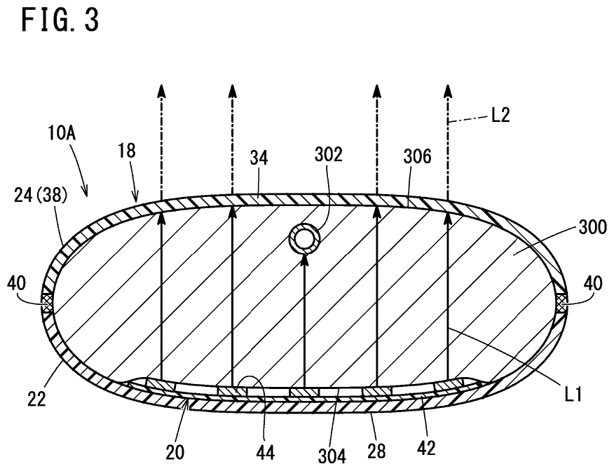

The blood vessel visualization device 10A visualizes a blood vessel 302 (see FIG. 3) in a site 300 to be visualized of a living body. In the present embodiment, the site 300 to be visualized is a human hand. Note that the site 300 to be visualized may be a region such as a forearm, an upper arm, a foot, a lower leg, or a thigh of a human body.

In FIGS. 1 to 3, the blood vessel visualization device 10A includes an attachment portion 18 and an irradiation unit 20. The attachment portion 18 is attachable to and detachable from the site 300 to be visualized. The attachment portion 18 has a shape corresponding to the site 300 to be visualized. Specifically, in the present embodiment, the attachment portion 18 has a glove shape covering each of the fingers, the palm, the back of the hand, and the wrist. The attachment portion 18 is divided into five fingers. The distal end of each finger of the attachment portion 18 in the fingertip direction is closed. A proximal end of the attachment portion 18 opposite to the fingertips is opened so that a user inserts or removes his/her hand into or from the attachment portion 18. The size and shape of the attachment portion 18 can be appropriately set.

In a case where the site 300 to be visualized is, for example, the foot, the attachment portion according to the present invention is formed in a shape covering the toes, the sole of the foot, the heel, the top of the foot, and the ankle. In this case, the attachment portion preferably has a shape divided into five fingers. Furthermore, in a case where the site 300 to be visualized is, for example, the forearm, the upper arm, the lower leg, or the thigh, the attachment portion according to the present disclosure is formed in a ring shape or a C shape.

In FIG. 3, the attachment portion 18 includes an attachment base 22 and a wavelength converter 24. In an attachment state in which the attachment portion 18 is attached to the site 300 to be visualized, an inner surface of the attachment base 22 covers a first surface 304 of the site 300 to be visualized. In the attachment state of the attachment portion 18, an inner surface of the wavelength converter 24 covers a second surface 306 of site 300 to be visualized that is on a side opposite to the first surface 304. In the attachment state of the attachment portion 18, the inner surface of the wavelength converter 24 is in contact (close contact) with the second surface 306 of the site 300 to be visualized. In the present embodiment, the first surface 304 is a surface on the palm side of the human hand, and the second surface 306 is a surface on the back side of the human hand.

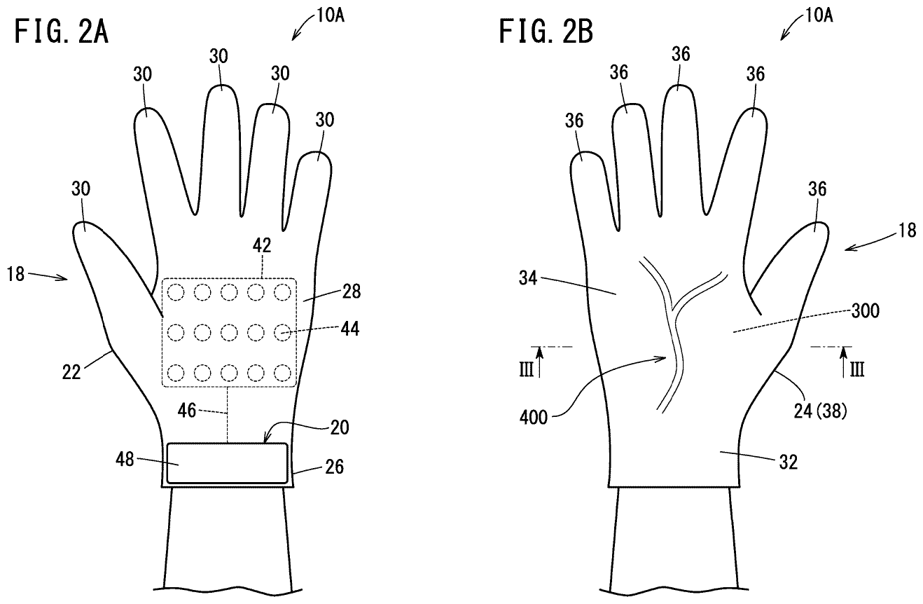

As illustrated in FIGS. 2A and 3, the attachment base 22 includes a wrist base 26, a palm base 28, and five finger bases 30. In the attachment state of the attachment portion 18, the wrist base 26 covers the inner surface of the wrist, the palm base 28 covers the palm, and the finger bases 30 cover the inner surfaces of the fingers, respectively. In the attachment state of the attachment portion 18, each of the finger bases 30 is in contact with (is in close contact with) the inner surface of the corresponding finger. The wrist base 26 is integrally connected to the palm base 28. The finger bases 30 protrude from and are integrally connected to the palm base 28.

The attachment base 22 is flexible. In this case, the attachment base 22 can be easily deformed in accordance with the shape of the site 300 to be visualized. Examples of the material of the attachment base 22 include a soft resin material. Note that the attachment base 22 may be formed of a hard resin material. The attachment base 22 cannot convert near-infrared light L1 into visible light L2.

As illustrated in FIGS. 2B and 3, the wavelength converter 24 includes a wrist cover 32, a back-of-hand cover 34, and five finger covers 36. In the attachment state of the attachment portion 18, an inner surface of the wrist cover 32 is in contact (close contact) with the outer surface of the wrist, the back-of-hand cover 34 is in contact (close contact) with the back of the hand, and the finger covers 36 are in contact (close contact) with the outer surfaces of the fingers, respectively. The wrist cover 32 is integrally connected to the back-of-hand cover 34. The finger covers 36 protrude from and are integrally connected to the back-of-hand cover 34. The wavelength converter 24 is flexible. In this case, the wavelength converter 24 can be easily deformed in accordance with the shape of the site 300 to be visualized.

The wavelength converter 24 includes a wavelength conversion material 38 (light upconversion material) that converts the near-infrared light L1 into the visible light L2. The wavelength conversion material 38 converts the near-infrared light L1 having a wavelength of more than 700 nm and 2500 nm or less into the visible light L2 having a wavelength of 400 nm or more and 700 nm or less. The wavelength conversion material 38 includes, for example, an inorganic light-upconversion light emitter or an organic light-upconversion light emitter. The inorganic light-upconversion light emitter contains, for example, a rare earth element. The organic light-upconversion light emitter includes, for example, an organometallic complex or a polycyclic aromatic compound.

The wavelength converter 24 is molded into a predetermined shape with a material containing the wavelength conversion material 38. Here, the “material containing the wavelength conversion material 38” includes a material containing only the wavelength conversion material 38 and a mixed material obtained by mixing another material with the wavelength conversion material 38. In the present embodiment, the wavelength converter 24 is molded into a predetermined shape only with the wavelength conversion material 38, for example. However, the wavelength converter 24 may be molded into a predetermined shape using a mixed material.

In FIGS. 1 and 3, the wavelength converter 24 is removably attached to the attachment base 22. The wavelength converter 24 is removably fixed to the attachment base 22 by a retainer 40. The retainer 40 is, for example, a fastener, a button, a hook, or the like.

The retainer 40 removably fastens the outer edge of the wavelength converter 24 to the outer edge of the attachment base 22. In other words, the retainer 40 fastens the outer edge of the wrist cover 32 to the outer edge of the wrist base 26. The retainer 40 also removably fastens the outer edge of the back-of-hand cover 34 to the outer edge of the palm base 28. The retainer 40 also removably fastens the outer edges of the finger covers 36 to the outer edges of the finger bases 30, respectively. In the attachment portion 18 described above, the wavelength converter 24 can be replaced with respect to the attachment base 22.

The attachment portion 18 may not have the plurality of finger bases 30 and the plurality of finger covers 36. In this case, each finger is exposed to the outside from the attachment portion 18 in the attachment state of the attachment portion 18.

As illustrated in FIGS. 2A and 3, the irradiation unit 20 includes a film portion 42, a plurality of light source units 44, a power supply line 46, and a power supply unit 48. The film portion 42 is fixed to the inner surface of the palm base 28. The film portion 42 is a polymer film and is flexible. The thickness of the film portion 42 is, for example, 1 μm or more and 10 μm or less. Note, however, that the thickness of the film portion 42 can be appropriately set.

Each of the light source units 44 emits near-infrared light L1 having a wavelength of more than 700 nm and 2500 nm or less. Each of the light source units 44 is, for example, an organic light-emitting diode (OLED). The organic light-emitting diode is deposited on the film portion 42. In this case, the film portion 42 and the plurality of light source units 44 can be easily bent, whereby the plurality of light source units 44 can be efficiently brought into close contact with the first surface 304 of the site 300 to be visualized.

Each of the light source units 44 is not limited to an organic light-emitting diode, and may be a so-called lamp light-emitting diode or chip light-emitting diode. The film portion 42 may be fixed to the outer surface of the palm base 28. In this case, the attachment base 22 is made of a material that transmits the near-infrared light L1. The number, arrangement, size, and shape of the light source units 44 can be appropriately changed.

The power supply line 46 supplies power of the power supply unit 48 to each of the light source units 44. The power supply line 46 connects the power supply unit 48 and the film portion 42 to each other. The film portion 42 is formed with a conductive pattern (not illustrated) for guiding the power supplied from the power supply line 46 to each of the light source units 44. The power supply line 46 is, for example, a cable embedded inside the attachment base 22. Note that the power supply line 46 may be fixed to the inner surface of the attachment base 22.

The power supply unit 48 is, for example, a primary cell or a secondary cell (battery). The power supply unit 48 is fixed to the attachment base 22. Specifically, the power supply unit 48 is fixed to the wrist base 26. The irradiation unit 20 may be configured to wirelessly supply power from the power supply unit 48 to each of the light source units 44. In this case, the power supply line 46 is unnecessary.

As illustrated in FIG. 1, the camera 14 captures a later-described blood vessel image 400 to be displayed on the wavelength converter 24. The camera 14 transmits the captured image to the information processing device 16. The camera 14 is connected to the information processing device 16 by wire.

The camera 14 may be wirelessly connected to the information processing device 16. Alternatively, the camera 14 may transmit the captured image to the information processing device 16 via an Internet line.

The information processing device 16 includes a calculation unit 50, a storage unit 52, a display unit 54, and a speaker 56. The calculation unit 50 is configured with a processor (processing circuit) such as a central processing unit (CPU) or a graphics processing unit (GPU).

The calculation unit 50 includes a control unit 58 and an image processing unit 60. The calculation unit 50 implements the control unit 58 and the image processing unit 60 by executing a program stored in the storage unit 52. Note that the calculation unit 50 may implement at least a part of the control unit 58 and the image processing unit 60 by an integrated circuit. Examples of the integrated circuit include an application specific integrated circuit (ASIC) and a field-programmable gate array (FPGA).

The storage unit 52 includes a volatile memory and a nonvolatile memory. Examples of the volatile memory include a random access memory (RAM). The volatile memory is used as a working memory of the processor, and temporarily stores data and the like necessary for processing or operation. Examples of the nonvolatile memory include a read only memory (ROM) and a flash memory. The nonvolatile memory is used as a storage memory. The nonvolatile memory stores programs, tables, maps, and the like. At least a part of the storage unit 52 may be incorporated in a processor or an integrated circuit as described above.

The control unit 58 controls the entire information processing device 16. The control unit 58 stores the image received from the camera 14 in the storage unit 52. The image processing unit 60 analyzes the image stored in the storage unit 52. Specifically, the image processing unit 60 extracts a change in images captured before and after a predetermined medical procedure, for example.

The display unit 54 displays an image received from the camera 14, an image analyzed by the image processing unit 60, and the like. The speaker 56 generates an alarm sound or the like.

Next, a method of using the blood vessel visualization system 12 will be described. The blood vessel visualization system 12 is used, for example, to detect a continuous or temporal change of the same blood vessel 302 at the site 300 to be visualized. Specifically, the blood vessel visualization system 12 is used, for example, to evaluate the effectiveness (effect of peripheral vasodilator and the like, effect of exercise therapy, and the like) of a medical procedure by comparing blood vessel images 400 of the blood vessel 302 that is a target before and after the medical procedure.

In the present embodiment, when the blood vessel visualization system 12 is used, the blood vessel visualization device 10A is prepared in which the wavelength converter 24 having, for example, a color suitable for the site 300 to be visualized (wavelength converter 24 having a color close to the color of the site 300 to be visualized) is attached to the attachment base 22. Thereafter, the attachment portion 18 of the blood vessel visualization device 10A is attached to the site 300 to be visualized (the left hand in FIG. 1 and the like) before a medical procedure is performed. At this time, the attachment base 22 and the wavelength converter 24 which are flexible are deformed so as to follow the shape of the site 300 to be visualized.

In a state in which the attachment of the attachment portion 18 to the site 300 to be visualized is completed, the plurality of light source units 44 is brought into close contact with the palm (the first surface 304 of the site 300 to be visualized). In addition, the back-of-hand cover 34 is in close contact with the back of the hand (the second surface 306 of the site 300 to be visualized). Further, the wrist cover 32 is in close contact with the outer surface of the wrist, the finger bases 30 are in close contact with the inner surfaces of the fingers, and the finger covers 36 are in close contact with the outer surfaces of the fingers. Therefore, the attachment portion 18 is fixed to the site 300 to be visualized by frictional resistance between the site 300 to be visualized and the attachment portion 18.

Thereafter, the power supply unit 48 is turned on. Thus, the near-infrared light L1 is emitted from the plurality of light source units 44 toward the second surface 306 of the site 300 to be visualized as illustrated in FIG. 3. The near-infrared light L1 passes through a region of the site 300 to be visualized other than the blood vessel 302. In other words, the near-infrared light L1 is absorbed by hemoglobin in the blood in the blood vessel 302 at the site 300 to be visualized. Transmitted light of the near-infrared light L1 transmitted through the site 300 to be visualized is converted into visible light L2 by the wavelength conversion material 38 of the wavelength converter 24. Therefore, a visible blood vessel image 400 is displayed on the wavelength converter 24 (see FIG. 2B).

Subsequently, the blood vessel image 400 displayed on the wavelength converter 24 is captured by the camera 14. The image (still image or moving image) captured by the camera 14 is transmitted to the information processing device 16 and stored in the storage unit 52. Hereinafter, an image captured before the execution of a medical procedure may be referred to as a “first image”. The first image may be stored in a storage unit of another computer via the Internet instead of being stored in the storage unit 52 of the information processing device 16. The control unit 58 determines whether or not the blood vessel image 400 is normal on the basis of the first image, for example, and generates an alarm sound from the speaker 56 when the blood vessel image is abnormal (for example, when blood flow may be obstructed by stenosis).

Thereafter, for example, a medical procedure for the patient is performed. After the medical procedure is performed, the blood vessel image 400 displayed on the wavelength converter 24 is captured by the camera 14 by the same method as described above. The image of the blood vessel image 400 after the medical procedure captured by the camera 14 is transmitted to the information processing device 16 and stored in the storage unit 52. Hereinafter, an image captured after the execution of the medical procedure may be referred to as a “second image”. The second image may be stored in a storage unit of another computer via the Internet instead of being stored in the storage unit 52 of the information processing device 16. The control unit 58 determines whether or not the blood vessel image 400 is normal on the basis of the second image, for example, and generates an alarm sound from the speaker 56 when the blood vessel image is abnormal (for example, when blood flow may be obstructed by stenosis).

Subsequently, the image processing unit 60 analyzes the first image and the second image to extract a changed portion between the first image and the second image. The control unit 58 displays the analysis result of the image processing unit 60 on the display unit 54. As a result, the user (including the patient) can easily and accurately know the effect of the medical procedure.

The present embodiment has the following effects.

According to the present embodiment, when the near-infared light L1 is emitted from the light source units 44 to the site 300 to be visualized in a state in which the attachment portion 18 is attached to the site 300 to be visualized, the visible blood vessel image 400 is displayed on the wavelength converter 24. That is, the blood vessel visualization device 10A can display the visible blood vessel image 400 on the wavelength converter 24 located on the site 300 to be visualized without using a device that converts the near-infrared light L1 into a blood vessel image. Therefore, the blood vessel visualization device 10A can have a simple and compact configuration.

In addition, the wavelength converter 24 can be fixed to the site 300 to be visualized in a state of being in contact with the second surface 306 of the site 300 to be visualized, whereby the near-infrared light LI transmitted through the site 300 to be visualized can be efficiently received by the wavelength converter 24. Therefore, the blood vessel image 400 can be more clearly displayed on the wavelength converter 24.

The attachment portion 18 includes the attachment base 22 that comes into contact with the first surface 304 in a state in which the attachment portion 18 is attached to the site 300 to be visualized.

With this configuration, the attachment base 22 facilitates fixing the attachment portion 18 to the site 300 to be visualized.

The wavelength converter 24 is removably attached to the attachment base 22.

With this configuration, only the wavelength converter 24 can be replaced when, for example, the wavelength converter 24 is damaged. As a result, the cost can be reduced as compared with the case where the entire blood vessel visualization device 10A is replaced. In addition, by preparing a plurality of wavelength converters 24 having different colors, it is possible to easily replace the wavelength converter 24 when it is desired to use the wavelength converter 24 in a color suitable for the site 300 to be visualized. Specifically, if the wavelength converter 24 having a color close to the color of the site 300 to be visualized is used, the wavelength converter 24 can be invisible with respect to the site 300 to be visualized when it is worn.

The light source units 44 are mounted on the attachment base 22.

With this configuration, the light source units 44 are arranged near the site 300 to be visualized, whereby the near-infrared light L1 can be efficiently emitted from the light source units 44 to the site 300 to be visualized.

The light source units 44 are mounted on the inner surface of the attachment base 22.

With this configuration, the light source units 44 can be brought into contact with (into close contact with) the first surface 304 of the site 300 to be visualized, whereby the near-infrared light L1 can be more efficiently emitted from the light source units 44 to the site 300 to be visualized.

The irradiation unit 20 includes the power supply unit 48 that supplies power to the light source units 44, and the power supply unit 48 is mounted on the attachment portion 18. With this configuration, the blood vessel visualization device 10A is compact and easy to carry, as compared with a case where the irradiation unit 20 is provided separately from the attachment portion 18.

The attachment portion 18 has a glove shape. With this configuration, the attachment portion 18 can be easily worn on the human hand.

The blood vessel visualization system 12 includes the camera 14 that captures the blood vessel image 400 displayed on the wavelength converter 24, and the image processing unit 60 that analyzes the image captured by the camera 14.

With this configuration, the camera 14 and the image processing unit 60 can acquire a change of the blood vessel image 400 displayed on the wavelength converter 24 after a lapse of a predetermined time.

### (Modification)

Next, a blood vessel visualization device 10B according to a modification will be described. In the present modification, the same components as those of the above-described blood vessel visualization device 10A are denoted by the same reference numerals, and the detailed description thereof will be omitted. In the present modification, the same effects are obtained for the same configuration as the blood vessel visualization device 10A.

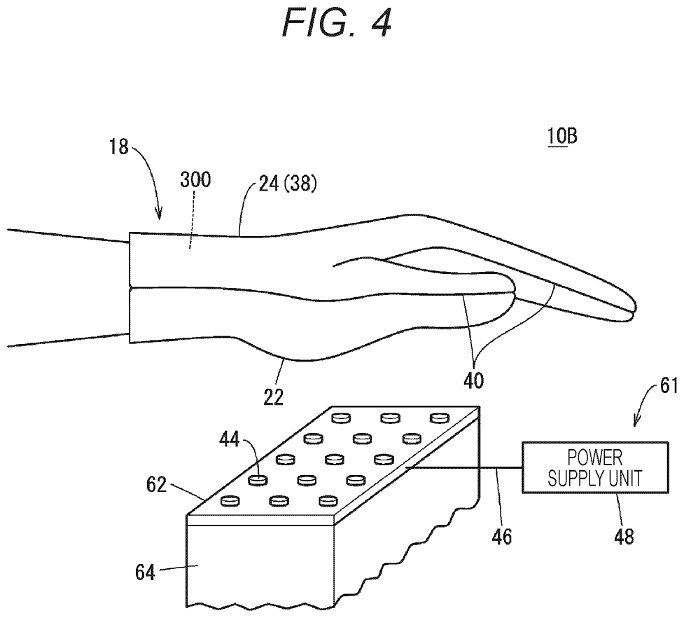

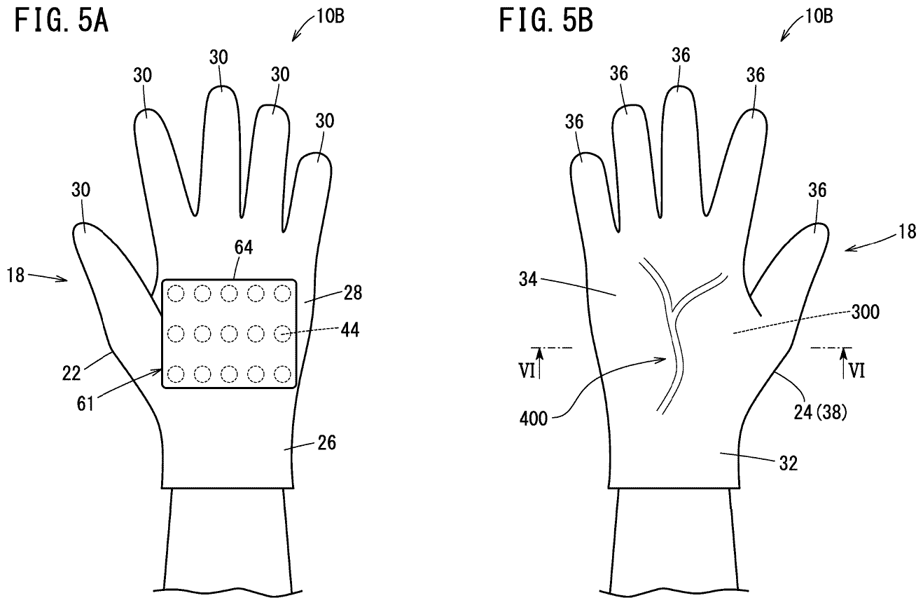

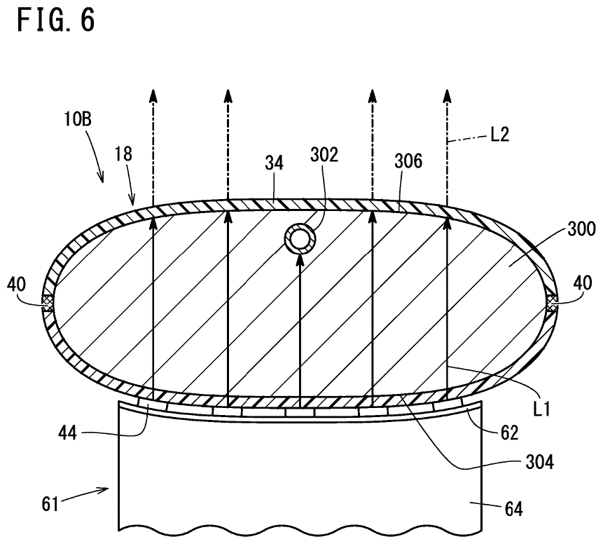

As illustrated in FIGS. 4 to 6, the blood vessel visualization device 10B includes an attachment portion 18 and an irradiation unit 61. The irradiation unit 61 is provided separately (as a separate component) from the attachment portion 18. The irradiation unit 61 includes a substrate 62, a plurality of light source units 44, an irradiation support unit 64, a power supply line 46, and a power supply unit 48. The substrate 62 is flexible. The light source units 44 are mounted on the substrate 62.

The light source units 44 are, for example, so-called chip light-emitting diodes. Note that the light source units 44 may be so-called lamp light-emitting diodes or the above-described organic light-emitting diodes. The irradiation support unit 64 supports the substrate 62 in a deformable manner. The power supply line 46 supplies power of the power supply unit 48 to each of the light source units 44.

In the present modification, the irradiation unit 61 is provided separately from the attachment portion 18.

This configuration eliminates the need to mount the irradiation unit 61 on the attachment portion 18, and thus, the blood vessel visualization device 10B can be easily manufactured.

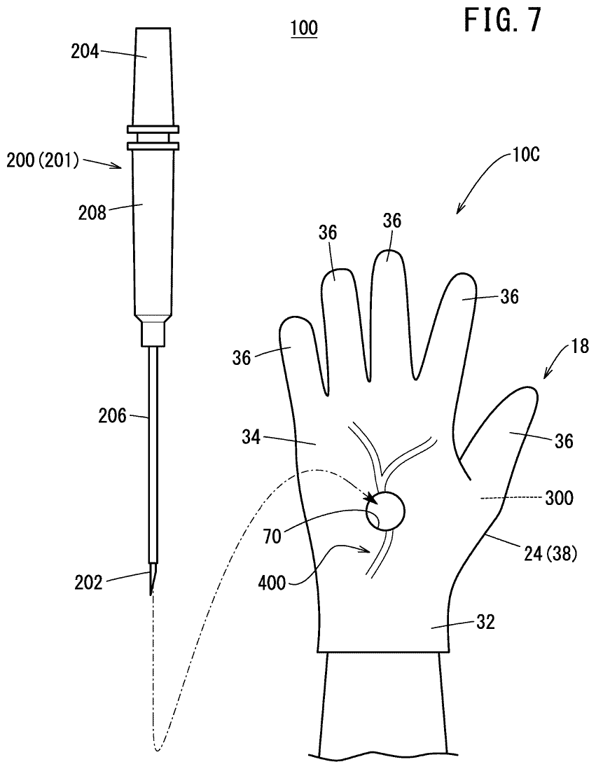

Next, a blood vessel puncture system 100 according to the present disclosure will be described. As illustrated in FIG. 7, the blood vessel puncture system 100 includes a blood vessel visualization device 10C and a medical device 200.

The blood vessel visualization device 10C is different from the blood vessel visualization device 10A described above in that a puncture hole 70 for puncturing the blood vessel 302 with the medical device 200 is formed in the wavelength converter 24. That is, the blood vessel visualization device 10C has the same configuration as the blood vessel visualization device 10A described above except for the puncture hole 70. Therefore, the components of the blood vessel visualization device 10C same as those of the blood vessel visualization device 10A described above are denoted by the same reference numerals, and the description thereof will be omitted.

The puncture hole 70 is located at, for example, a position including a region where the blood vessel 302 (a radial artery, for example) to be punctured is assumed to be located in a state in which the attachment portion 18 is attached to the site 300 to be visualized. Specifically, the puncture hole 70 is located, for example, at a central part of the back-of-hand cover 34. In the present embodiment, the puncture hole 70 is a circular hole. The diameter of the puncture hole 70 is set to, for example, 1 mm or more and 10 mm or less. Note that the shape, position, and size of the puncture hole 70 can be appropriately set.

The medical device 200 is, for example, a catheter assembly 201. The catheter assembly 201 includes a needle body 202, a needle hub 204, a catheter shaft 206, and a catheter hub 208. The needle body 202 is configured to puncture the blood vessel 302. The needle hub 204 is provided at a proximal end of the needle body 202. The catheter shaft 206 extends in a tubular shape. In an initial state, the needle body 202 is inserted into the lumen of the catheter shaft 206. The catheter hub 208 is provided at a proximal end of the catheter shaft 206. The medical device 200 is not limited to the catheter assembly 201, and may be a puncture needle for blood sampling or the like.

The blood vessel visualization device 10C may have a plurality of puncture holes 70. In this case, the number and position of the puncture holes 70 can be appropriately set.

The blood vessel puncture system 100 has the following effects.

According to the blood vessel puncture system 100, the blood vessel 302 can be punctured with the medical device 200 (catheter assembly 201) with the blood vessel image 400 displayed on the wavelength converter 24 being visually recognized, so that the blood vessel can be smoothly punctured with the medical device 200.

The wavelength converter 24 has the puncture hole 70 for puncturing the blood vessel 302 with the medical device 200.

With this configuration, the medical device 200 can puncture the blood vessel 302 through the puncture hole 70. As a result, the puncture resistance can be reduced as compared with a case of penetrating the medical device 200 through the wavelength converter 24. In addition, it is possible to reduce the possibility that the wavelength converter 24 intrudes into the site 300 to be visualized as a foreign substance.

The blood vessel puncture system 100 may include the blood vessel visualization device 10A or the blood vessel visualization device 10B described above instead of the blood vessel visualization device 10C. Furthermore, the blood vessel puncture system 100 may include the camera 14 and the information processing device 16 described above.

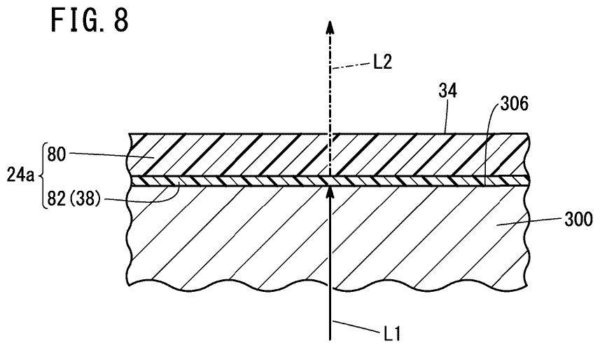

The blood vessel visualization devices 10A to 10C may include a wavelength converter 24a according to a modification illustrated in FIG. 8 instead of the wavelength converter 24 described above. The wavelength converter 24a includes a base 80 and a coating 82. The base 80 is made of a material not containing the wavelength conversion material 38. The coating 82 is formed by coating an inner surface of the base 80 (a surface facing the second surface 306 of the site 300 to be visualized) with the wavelength conversion material 38.

The coating 82 may be formed by coating an outer surface of the base 80 (a surface facing the side opposite to the site 300 to be visualized) with the wavelength conversion material 38. The coating 82 may be provided on both the inner surface and the outer surface of the base 80.

The wavelength converters 24 and 24a and the attachment base 22 may be integrally molded. That is, the wavelength converters 24 and 24a may be provided integrally with the attachment base 22 so as not to be removable from the attachment base 22. In this case, the blood vessel visualization devices 10A to 10C can be easily manufactured.

Note that the present invention is not limited to the above-described embodiment, and various configurations can be adopted without departing from the gist of the present invention.

What is claimed is:

1. A blood vessel visualization device for visualizing a blood vessel of a living body in a site to be visualized that includes a first surface and a second surface on a side opposite the first surface, the blood vessel visualization device comprising:
an attachment portion attachable to and detachable from the site to be visualized; and
an irradiation unit comprising a light source unit for irradiating the first surface with near-infrared light, wherein:
the attachment portion comprises a wavelength converter comprising a wavelength conversion material that converts the near-infrared light into visible light, the attachment portion being fixable to the site to be visualized in a state in which the wavelength converter is in contact with the second surface.

2. The blood vessel visualization device according to claim 1, wherein:
the attachment portion comprises an attachment base configured to contact the first surface in a state in which the attachment portion is attached to the site to be visualized.

3. The blood vessel visualization device according to claim 2, wherein:
the wavelength converter is removably mounted on the attachment base.

4. The blood vessel visualization device according to claim 2, wherein:
the light source unit is mounted on the attachment base.

5. The blood vessel visualization device according to claim 4, wherein:
the light source unit is mounted on an inner surface of the attachment base.

6. The blood vessel visualization device according to claim 4, wherein:
the irradiation unit comprises a power supply unit that supplies power to the light source unit, and
the power supply unit is mounted on the attachment portion.

7. The blood vessel visualization device according to claim 1, wherein:
the irradiation unit is separate from the attachment portion.

8. The blood vessel visualization device according to claim 1, wherein:
the wavelength converter is made of a molded material containing the wavelength conversion material.

9. The blood vessel visualization device according to claim 1, wherein:
the wavelength converter comprises:
a base made of a material not containing the wavelength conversion material; and
a coating made of the wavelength conversion material, coated on a surface of the base.

10. The blood vessel visualization device according to claim 1, wherein:
the wavelength conversion material converts the near-infrared light having a wavelength of more than 700 nm and 2500 nm or less into the visible light having a wavelength of 400 nm or more and 700 nm or less.

11. The blood vessel visualization device according to claim 1, wherein:
the wavelength converter comprises a puncture hole through which the blood vessel is puncturable with a medical device.

12. The blood vessel visualization device according to claim 1, wherein:
the attachment portion has a glove shape.

13. A blood vessel puncture system comprising:
the blood vessel visualization device according to claim 1; and
a medical device configured to puncture the blood vessel.

14. A blood vessel visualization system comprising:
a blood vessel visualization device for visualizing a blood vessel of a living body in a site to be visualized that includes a first surface and a second surface on a side opposite the first surface, the blood vessel visualization device comprising:
an attachment portion attachable to and detachable from the site to be visualized; and
an irradiation unit comprising a light source unit for irradiating the first surface with near-infrared light, wherein:
the attachment portion comprises a wavelength converter comprising a wavelength conversion material that converts the near-infrared light into visible light a camera configured to capture a blood vessel image displayed on the wavelength converter; and
an image processing unit configured to analyze an image captured by the camera.

15. A method of capturing a visible blood vessel image of a blood vessel in a site to be visualized that includes a first surface and a second surface on a side opposite the first surface, the method comprising:
providing a blood vessel visualization device comprising:
an attachment portion, and
an irradiation unit comprising a light source unit, wherein:
the attachment portion comprises a wavelength converter comprising a wavelength conversion material that converts the near-infrared light into visible light;
attaching the attachment portion to the site to be visualized such that the wavelength converter is in contact with the second surface;
emitting near-infrared light from the irradiation unit toward the first surface of the site to be visualized; and
capturing, by a camera, a visible blood vessel image that is displayed on the wavelength converter.

16. A method of detecting a change in a blood vessel of a patient, the method comprising:
performing the method of claim 15 a first time to capture a first visible blood vessel image;
performing a medical procedure on the patient;
performing the method of claim 15 a second time to capture a second visible blood vessel image; and
analyzing, using an image processing unit, the first visible blood vessel image and the second visible blood vessel image, to determine an effect of the medical procedure.
